(19) United States
(12) Patent Application Publication
(10) Pub. No.: US 2015/0056173 A1
(43) Pub. Date: Feb. 26, 2015

(54) BETA CELL GROWTH AND DIFFERENTIATION

(71) Applicants: Joslin Diabetes Center, Inc., Boston, MA (US); The Regents of the University of California, Oakland, CA (US)

(72) Inventors: Rohit N. Kulkarni, Brookline, MA (US); Ulupi S. Jhala, San Diego, CA (US)

(21) Appl. No.: 14/478,340

(22) Filed: Sep. 5, 2014

Related U.S. Application Data
(63) Continuation of application No. 11/429,164, filed on May 5, 2006.
(60) Provisional application No. 60/678,324, filed on May 6, 2005.

(51) Int. Cl. A61K 35/39; C12N 5/071

(52) U.S. Cl. CPC A61K 35/39; C12N 5/0676; C12N 2501/599
USPC 424/93.7; 435/366; 435/377

(57) ABSTRACT

The invention includes methods that can be used to increase β cell populations in vivo and in vitro, useful in the treatment of diabetes and related disorders.

### CLAIM OF PRIORITY

This application claims the benefit under 35 USC §119(e) of U.S. Provisional Patent Application Ser. No. 60/678,324, filed on May 6, 2005, the entire contents of which are hereby incorporated by reference.

### FEDERALLY SPONSORED RESEARCH OR DEVELOPMENT

This invention was made with Government support under Grant No. DK67536 awarded by the National Institutes of Health. The Government has certain rights in the invention.

### TECHNICAL FIELD

This invention relates to methods and compositions for enhancing pancreatic beta cell growth and differentiation.

### BACKGROUND

Islet hyperplasia and hyperinsulinemia develop to varying degrees in virtually all states of insulin resistance and are apparent in humans, rodents, and other mammals in the presence of obesity, genetic insulin resistance, and states of stress or when counterinsulin hormones are chronically elevated (reviewed in Kulkarni and Kahn, 2001. “Genetic models of insulin resistance: alterations in β-cell biology.” In Molecular basis of pancreas development and function. J. F. Habener and M. Hussain, editors. Kluwer Academic Publishers. New York, N.Y., USA. 299-323). The factors that stimulate growth, the specific proteins in the β cells, and the precise mechanisms that regulate this compensatory hyperplasia in insulin resistance remain poorly defined (Id.). The pancreatic homeodomain protein PDX-1 and the insulin/insulin-like growth factor I (IGF-I) signaling pathway are both important for growth and cell proliferation in the pancreas. While PDX-1 has been shown to regulate expansion of pancreatic progenitor cells (reviewed in Melloul et al., 2002. Diabetologia. 45:309-326), proteins in the insulin/IGF-1 signaling pathway are known to regulate growth, cell proliferation, adhesion, and tissue architecture as well as to modulate metabolism in virtually all tissues in mammals, including the pancreatic islets (Cheatham and Kahn, 1995. Endocr. Rev. 16:117-142; Potter et al., 1999. Endocr. Rev. 20:207-239).

PDX-1 regulates target gene transcription both as a monomer and as a heterodimer with the three amino acid loop extension homeodomain protein PBX-1. The monomeric and dimeric forms of PDX regulate specific and distinct targets. PDX-PBX dimerization has been shown to be critical for embryonic pancreatic cell proliferation (Dutta et al., 2001. Proc. Natl. Acad. Sci. U.S.A. 98:1065-1070; Asahara et al., 1999. Mol. Cell. Biol. 19:8219-8225; Kim et al., 2002. Nat. Genet. 30:430-435).

PBX-1—null mice display an approximately 30% decrease in pancreatic cell proliferation, while mice expressing the PBX-1 interaction-deficient PDX-1, on a PDX-null background, manifest a severe attenuation of pancreatic cell expansion during embryogenesis (Kim et al., 2002. Nat. Genet. 30:430-435). Mice with PDX-1 heterozygosity have been reported to exhibit enhanced β cell apoptosis (Johnson et al., 2003. J. Clin. Invest. 111:1147-1160). On the other hand, mice with a β cell-specific KO of the insulin receptor (IR) exhibit decreased islet growth in adults and a susceptibility to developing overt diabetes (Kulkarni et al., 1999. Cell. 96:329-339; Otani et al., 2004. Am. J. Physiol. Endocrinol. Metab. 286:E41-E49). Although mice with IR substrate-1 (IRS-1) deficiency show attenuated nutrient sensing in the islets, the compensatory islet hyperplasia in response to insulin resistance is maintained (Kulkarni et al., 1999. J. Clin. Invest. 104:R69-R75; Kubota et al., 2000. Diabetes. 49:1880-1889). Similarly, mice double heterozygous for the IR and IRS-1 (also called IR/IRS-1 mice) and liver-specific IR KO (LIRKO) mice both have severe insulin resistance that results in massive compensatory hyperplasia with up to a 10-fold increase in β cell mass (Bruning et al., 1997. Cell. 88:561-572; Michael et al., 2000. Mol. Cell. 6:87-97). In contrast, deficiency of IRS-2 results in hyperglycemia, β cell apoptosis (Kubota et al., 2000. Diabetes. 49:1880-1889; Withers et al., 1998. Nature. 391:900-904; Kitamura et al., 2002. J. Clin. Invest. 110:1839-1847), and a strain-dependent dysregulation of PDX-1 expression (Suzuki et al., 2003. J. Biol. Chem. 278:43691-43698).

### SUMMARY

The present invention is based, at least in part, on the discovery of mechanisms of growth and proliferation of adult β cells in insulin-resistant states, and their importance in the maintenance of β cell mass. Described herein are methods that can be used to increase β cell populations in vivo and in vitro. The methods include providing a population of differentiated β cells, inducing the cells to de-differentiate, allowing the de-differentiated cells to proliferate, and allowing the cells to re-differentiate into glucose-sensitive, insulin-secreting β cells.

In one aspect, the invention includes methods for increasing an initial population of mammalian β-cells that secrete insulin in response to glucose. The methods include providing an initial population of fully-differentiated β-cells from a mammal, e.g., a postnatal, juvenile, adolescent, or adult mammal, e.g., a human; contacting the cells with, e.g., administering or culturing the cells in the presence of, a modulator e.g., an exogenous modulator, of E-cadherin/β-catenin signaling, e.g., a compound that (i) inhibits E-cadherin and/or (ii) enhances β-catenin signalling, in an amount and for a time sufficient to cause the cells to de-differentiate, i.e., to undergo an epithelial to mesenchymal type transition, e.g., to a less-differentiated morphological state, wherein the de-differentiated cells do not secrete (e.g., detectably or substantially secrete) insulin in response to glucose; allowing the de-differentiated cells to proliferate (e.g., for a time sufficient to increase the population); and removing the modulator, e.g., by culturing/incubating the de-differentiated cells in the absence of the modulator, or reducing the concentration or amount of the modulator, to allow the de-differentiated cells to re-differentiate into β-cells that secrete insulin; thereby increasing the initial population of mammalian β-cells that secrete insulin in response to glucose.

In another aspect, the invention provides methods for providing a population of mammalian β-cells that secrete insulin in response to glucose. The methods include providing at least one fully-differentiated β-cell from a mammal, e.g., an adult mammal, e.g., a human; contacting the cell with, e.g., culturing/incubating the cell in the presence of, a modulator, e.g., an exogenous modulator, of E-cadherin/β-catenin signaling, e.g., a compound that (i) inhibits E-cadherin and/or (ii) enhances β-catenin signalling, in an amount and for a time sufficient to cause the cell to undergo an epithelial to mesenchymal type transition, e.g., transition to a less-differentiated morphological state, wherein the cell does not secrete insulin in response to glucose, e.g., does not detectably or substantially secrete glucose; allowing the cell to proliferate for a time sufficient to produce a desired population of cells; and removing the modulator, e.g., by culturing the population of cells in the absence of the modulator, or reducing the concentration or amount of the modulator, to allow the population of cells to re-differentiate into β-cells that secrete insulin; thereby providing a population of mammalian β-cells that secrete insulin in response to glucose.

In some embodiments, the methods include one or more of determining if the re-differentiated β-cells secrete insulin, e.g., secrete insulin in a glucose-dependent manner; placing the re-differentiated cells into a sterile preparation, e.g., a preparation comprising a therapeutically effective number of cells or a portion thereof, e.g., about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more islet equivalents; and returning the re-differentiated cells to the mammal from which they came, or transplanting the re-differentiated cells to another mammal, e.g., of the same or different species. In some embodiments, a mean (±SD) islet mass of at least about 10,000 islet equivalents per kilogram of body weight is transplanted.

In some embodiments, the modulator is selected from the group consisting of antibodies that bind selectively to E-cadherin; E-cadherin dominant negatives; constitutively active forms of beta-catenin, and activators of the Wnt signaling pathway.

In some embodiments, the β cell or initial population of mammalian β-cells is in the pancreas of a living mammal, e.g., a human, wherein contacting the cells with the modulator comprises administering a therapeutic composition comprising the modulator to the mammal, e.g., locally into pancreas. In some embodiments, the cell or cells are derived from or in a human.

In another aspect, the invention features methods for increasing a population of glucose-sensitive insulin secreting cells in a subject. The methods include transplanting a population of re-differentiated cells produced by a method described herein into the subject, e.g., wherein the cells were originally derived from the subject.

As used herein, a cell that is “derived from” an animal is a cell that was taken from the animal, or a cell that is a progeny cell of a progenitor cell that was taken from the animal, e.g., removed from the animal surgically or by some other method.

In another aspect, the invention features methods for increasing a population of glucose-sensitive insulin secreting cells in a subject. The methods include transiently (e.g., for a limited time) administering to the subject one or more doses of a modulator, e.g., an exogenous modulator, of E-cadherin/β-catenin signaling, e.g., a compound that (i) inhibits E-cadherin and/or (ii) enhances β-catenin signalling, e.g., locally into pancreas. In some embodiments, the methods include monitoring the growth of cells in the pancreas of the subject, and stopping (or reducing) the administration of the modulator when there is a sufficient number of cells. In some embodiments, the methods include administration of the modulator locally to the pancreas.

De-differentiation mean a transition from a more differentiated state to a less differentiated state; this is also referred to herein as an epithelial to mesenchymal type transition. As used herein, a cell that is de-differentiated is a cell that has lost the ability to secrete insulin in a glucose-regulated manner, and has a morphology that resembles a more primitive cell type, e.g., a mesenchymal morphology. A fully differentiated cell, conversely, can secrete insulin in a glucose-regulated manner, has a β cell type morphology, and is capable of forming adherens junctions. See, e.g., Volk et al., Arch Pathol. 88(4):413-22 (1969).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a pair of line graphs illustrating changes in body weight of male mice between the ages of 4 and 20 weeks. Mice are divided into IR/IRS-1 (left panel) and PDX-1 (right panel) groups. P<0.05, IRS-1 or IR/IRS-1 vs. WT at all time points; P<0.05, IR vs. WT from 12 weeks onward; P<0.05, IRS-1/PDX-1 or IR/IRS-1/PDX-1 triple heterozygous KO (TKO) vs. PDX-1 at all time points, IR/PDX-1 vs. PDX-1 from 12 weeks onward (n=12-26).

FIG. 1B is a bar graph illustrating fasting blood glucose measured after a 14-hour overnight fast in 2- and 4-month-old male mice.

FIG. 1C is a bar graph illustrating fed blood glucose measured in random-fed 2- and 4-month-old male mice.

FIG. 1D is a bar graph illustrating fasting serum insulin was measured after a 14-hour overnight fast in 2- and 4-month-old male mice.

FIG. 1E is a bar graph illustrating C-peptide levels measured by RIA.

*P<0.05, IR/PDX-1, IRS-1/PDX-1, or TKO vs. PDX-1; †P<0.05, IR, IR/IRS-1, or PDX-1 vs. WT (n=10-22).

FIGS. 2A and 2B are line graphs illustrating the acute phase insulin secretory response to intraperitoneal (i.p.) injection of glucose, measured in mice after a 14-hour overnight fast. *P<0.05, WT vs. PDX-1, IR/PDX-1, IRS-1/PDX-1, or TKO; _P<0.05, IRS-1 or IR/IRS-1 vs. WT (n=4_{—}10).

FIGS. 2C-2F are line graphs illustrating glucose tolerance after intraperitoneal injection of glucose, measured after a 14-hour fast in 2-month-old (2C and 2D) and 4-month-old (2E and 2F) male mice. In 2-month-old mice, *P<0.05, IRS-1/PDX-1 or TKO vs. PDX-1; P<0.05, PDX-1 vs. WT. In 4-month-old mice, *P<0.05, IRS-1/PDX-1 or TKO vs. WT; P<0.05, IR/IRS-1 or PDX-1 vs. WT (n=7-18).

FIG. 3A is a reproduction of a Western blot of PDX-1 and CREB proteins prepared from pancreas as described in Methods. Each lane corresponds to an individual mouse (n=3). FIG. 3B is a bar graph illustrating quantitation of PDX-1 levels in 3A. Data are depicted as the ratio of PDX-1 to CREB and expressed as arbitrary densitometry units. *P<0.05, PDX-1 vs. IRS-1/PDX-1 or TKO; P<0.05, PDX-1 or IR/IRS-1 vs. WT (n=3).

FIG. 3C is a reproduction of a Western blot of PDX-1 and CREB proteins prepared from freshly isolated islets from WT and IR/IRS-1 mice (n=2).

FIG. 3D is a photomicrograph of representative pancreas sections stained with cocktail antibody to non-β cell hormones as described in Methods. Scale bar: 50 μm.

FIG. 3E is a bar graph illustrating β Cell mass estimated by morphometric analysis as described in Methods. *P<0.05, IR/PDX-1, IRS-1/PDX-1, or TKO vs. PDX-1; P<0.05, IR, IRS-1, or IR/IRS-1 vs. WT.

FIG. 3F is a bar graph illustrating pancreatic insulin content measured in acid-ethanol extracts of homogenized pancreas as described in Methods. *P<0.05, IRS-1/PDX-1 or TKO vs. PDX-1; P<0.05, IR/IRS-1 vs. WT (n=4-6).

FIG. 4A is a panel of 21 photomicrographs of representative islets from pancreas sections stained with immunofluorescent antibodies for insulin (upper panels, green), glucagon (middle panels, green), synaptophysin (middle panels, red), and Glut2 (lower panels, red) as described in Methods.

FIG. 4B is a panel of eight photomicrographs of representative islets from pancreas sections from the TKO group, costained for insulin (purple), glucagon (green), and somatostatin (red). Four different TKO islets are shown.

FIGS. 5A-5D are photomicrographs of cells immunostained for pancreatic ductal marker, and markers for cell death and neogenesis. FIGS. 5A and 5B are each seven representative sections of pancreas from different genotypes, showing islets stained for PCNA (5A) and caspase-3 (5B). Note the magnified image for TKO. FIG. 5C shows consecutive sections of pancreas from IR/IRS-1 mice immunostained for PCNA (left panels) or costained for insulin (green) and DBA-lectin immunohistochemistry for duct-specific glycoconjugates (red) (upper right panel). The lower right panels show 2 magnified images (×60) of islet cells from IR/IRS-1 mice; the arrows point to cells positive for PCNA (purple chromogen) that also stain positive for nuclear β-catenin (blue chromogen). FIG. 5D shows Pancreas sections from WT, IR/IRS-1, PDX-1 heterozygous, IRS-1/PDX-1, and TKO mice costained for E-cadherin (purple) and (β-catenin (orange) as described in Methods. Scale bars: 50 μm.

FIG. 6A is a bar graph illustrating changes in β Cell mass in the LIRKO mouse estimated by morphometric analysis as described in Methods. *P<0.05, LIRKO vs. IRLox (control), PDX-1, or LIRKO/PDX-1; P<0.05, LIRKO/PDX-1 vs. IRLox or PDX-1 (n=4_{—}6).

FIG. 6B is a set of four photomicrographs of representative islets from pancreas sections from the LIRKO group, costained for insulin (purple), glucagon (green), and somatostatin (red).

FIG. 6C is a set of four photomicrographs of representative pancreas sections from IRLox, PDX-1, LIRKO, and LIRKO/PDX-1 mice, costained for E-cadherin (purple) and β-catenin (orange) as described in Methods. Scale bars: 50 μm

### DETAILED DESCRIPTION

In the experiments described herein, 2 independent models of insulin resistance were used to demonstrate that β cell replication occurs even in postdevelopmental states of β cell growth. The lineage tracing of β cells and the mechanism of replication of adult β cells are currently areas of intense research. While ductal cells were previously thought to be the sole precursors of islet neogenesis, in the two models examined in this study, the proliferating PCNA+ cells in the actively expanding islet were negative for a ductal marker. Furthermore, the fact that the associated changes in the composition of the adherens junction occurred only in large islets in the insulin-resistant mice indicates that a coordinated and regulated process of beta cell replication, consistent with a process analogous to an epithelial-mesenchymal transition, also plays a role in beta cell maintenance of beta cell mass.

Methods for Obtaining Pancreatic Beta Cells

The methods described herein can be used to increase or provide a population of fully-differentiated β cells, e.g., derived from a living mammal, or cultured cells. This can include autologous β cells, i.e., a cell or cells taken from a subject who is in need of additional β cells (i.e., the donor and recipient are the same individual). This has the advantage of avoiding any immunologically-based rejection of the cells. Alternatively, the cells can be heterologous, e.g., taken from a donor. The second subject can be of the same or different species. Typically, when the cells come from a donor, they will be from a donor who is sufficiently immunologically compatible with the recipient, i.e., will not be subject to transplant rejection, to lessen or remove the need for immunosuppression. In some embodiments, the cells are taken from a xenogeneic source, i.e., a non-human mammal that has been genetically engineered to be sufficiently immunologically compatible with the recipient, or the recipient's species. Methods for determining immunological compatibility are known in the art, and include tissue typing to assess donor-recipient compatibility for HLA and ABO determinants. See, e.g., Transplantation Immunology, Bach and Auchincloss, Eds. (Wiley, John & Sons, Incorporated 1994).

In some embodiments, the fully-differentiated β cells are in a stabilized state, e.g., the cells were taken from a mammal and treated in such a manner as to allow them to be stored for some period of time. For example, the cells can be frozen, e.g., using methods known in the art for freezing primary cells, such that the cells are viable when thawed. For example, methods known in the art to freeze and thaw embryos to generate live mammals can be adapted for use in the present methods. Such methods may include the use of liquid nitrogen, e.g., with one or more cryoprotectants, e.g., agents that prevent freeze-thaw damage to the cell. In some embodiments, the cells were de-differentiated and proliferated using a method described herein before being stabilized (e.g., frozen); in some embodiments, the cells were also re-differentiated before being stabilized. The invention also includes populations of cells, e.g., cells in a stabilized state, that are made by this method. In some embodiments, the invention includes a preparation comprising a therapeutically effective number of stabilized cells or a portion thereof, e.g., about 1×10⁶, 2×10⁶, 3×10⁶, 4×10⁶, 5×10⁶, 6×10⁶, 7×10⁶, 8×10⁶, 9×10⁶, 1×10⁷, 2×10⁷, or more cells.

Methods for surgically removing and transplanting suitable fully-differentiated β cells from a mammal are known in the art; see, e.g., Shapiro et al., N Engl J. Med. 343(4):230-8 (2000); Ryan et al., Diabetes 50(4):710-9 (2001).

The population of fully-differentiated β cells will typically be substantially pure, e.g., not more than about 40% undifferentiated cells, i.e., at least about 60% fully differentiated β cells. In some embodiments, the population is at least about 70%, 75%, 80%, 90%, 95% or more fully-differentiated β cells. The purity of the population can be determined, and manipulated, using methods known in the art. For example, methods using fluorescence activated cell sorting can be used. For example, duct epithelial cells can be detected and counted, e.g., by labeling the cells with a fluorescence-labeled duct-specific lectin (e.g., Dolichos biflorus agglutinin (DBA)), as described herein, and removed from the population, e.g., by fluorescence-activated cell sorting methods (e.g., flow sorting) or immunosorbtion to a substrate, such as a column or beads, bound to DBA. Other non-β cells can be removed using similar methods, including flow sorting based on autofluorescence. Fully-differentiated β cells can be detected and counted, e.g., by labeling the cells with a fluorescent-labeled antibody to a β cell marker, such as insulin or E-cadherin or other beta cell surface marker, e.g., as described in Zhang et al., Diabetes. 50(10):2231-6 (2001). In some embodiments, the use of an antibody that does not significantly alter the β cells is desired.

Modulators of E-Cadherin/β-Catenin Signalling

The methods described herein include contacting the cells with one or more modulators of E-cadherin/β-catenin signalling, to induce the cells to de-differentiate (e.g., to induce an epithelial-to-mesenchymal type transition), then removing the modulator when the cells have reached a desired population number or density. In some embodiments, the modulator affects one or more of the following:
1. E-cadherin: This is an important adhesion protein involved in maintaining cell-cell adhesion. Without wishing to be bound by theory, it is likely that the first step in the EMT process involves a decrease in the expression of E-cadherin. Thus, compounds that inhibit E-cadherin signalling would be suitable for use in the methods described herein. Such compounds include anti-E-cadherin antibodies and antigen-binding fragments thereof, e.g., the rat monoclonal antibody DECMA-1, as described in Vestweber and Kemler, EMBO J. 4(13A):3393-8 (1985); Nakagawa et al., J Cell Sci. 114(10):1829-38 (2001); other anti-E-cadherin antibodies are commercially available, e.g., from Takara Shuzo Biomedicals Co., Ltd. (Shiga, Japan; a SHE78-7 murine IgG2a monoclonal antibody); Santa Cruz Biotechnology (Santa Cruz, Calif.), PanVera (Madison, Wis.), and BD Biosciences (San Jose, Calif.).
   Compounds that inhibit E-cadherin signalling by decreasing E-cadherin levels are also suitable for use in the methods described herein. For example, slug or compounds that induce slug, which is an inhibitor of E-cadherin transcription, can be used, as can E-cadherin specific siRNA, aptamer, or antisense.
2. β-catenin: This protein is associated with E-cadherin, and translocation of this protein to the nucleus allows it to regulate genes involved in proliferation and replication. Compounds that enhance β-catenin signaling are suitable for use in the present methods. For example, compounds that increase β-catenin expression, or enhance β-catenin translocation to the nucleus, are suitable. Constitutively active forms of beta-catenin can also be used, e.g., as described in Furlong et al., Gynecol. Oncol. 77(1):97-104 (2000), and Yost et al., Genes Dev. 10:1443-1454 (1996).
3. GSK3: This protein is part of the Wnt signaling pathway and may play a role in protein synthesis. Other members of the Wnt pathway can also be targeted. See, e.g., Moon RT. Science STKE, 2005, DOI: 10.1126/stke.2712005 cm1.
4. Snail/slug/slit family of transcription factors: These factors are known to be involved in gene regulation in the EMT process in other cell types.

Once the cells have de-differentiated, they will proliferate. The cells can be monitored to determine whether any changes, e.g., changes in genetic makeup, have occurred during time in culture. The cells can also be monitored to determine population numbers. In some embodiments, the cells are maintained in a commercially available medium, e.g., RPMI or DMEM (see, e.g., Hamid et al., Cell Transplantation, 10(2):153-159(7) (2001).

Once the cells have reached a desired population number, the modulator can be removed, i.e., concentrations are reduced, or the modulator is no longer provided or administered. In the absence of the modulator, the cells should re-differentiate into insulin-secreting β cells. Re-differentiation can be monitored, e.g., using insulin secretion as a marker. At this point, the re-differentiated cells can be suspended in a media suitable for administration into a subject, or stabilized for storage. Alternatively, the cells can be stabilized before re-differentiation occurs, e.g., in the presence or absence of the modulator of E-cadherin/β-catenin signalling. The invention also includes stabilized populations of cells prepared by a method described herein.

PDX-1

As described herein, PDX-1 is an important regulator of β cell replication. PDX-1 is linked to β cell neogenesis in the NOD mouse and in a model of pancreatic injury (partial-pancreatectomy), and is associated with increased proliferation of isolated duct cells (O'Reilly, L. A. et al. 1997. Diabetes. 46:599-606; Sharma et al., 1999. Diabetes. 48:507-513).

Although the specific molecular mechanism(s) by which PDX-1 modulates β cell replication is not fully understood, the low expression of β-catenin in β cells in the PDX-1 islet and the nuclear translocation of β-catenin in islet hyperplasia in the IR/IRS-1 mice suggest an association between PDX-1 and β-catenin in the coordination of β cell expansion. A recent study reporting that β cells in adult mice are generated from preexisting β cells (Dor et al., 2004. Nature. 429:41-46) favors replication as the process most actively involved in the compensatory response to insulin resistance.

The profound decrease in p cell mass due to apoptosis in the IR/IRS-1/PDX-1 triple heterozygous KO (TKO) group was unexpected. Studies on differentiated neuronal cells (Becker and Bonni, 2004. Prog. Neurobiol. 72:1-25) suggest that apoptosis is a potential default mechanism secondary to an abortive attempt at entering the cell cycle. Such a hypothesis is consistent with the increased caspase-3 staining in TKO islets, and the β cell death in these mice may indeed be secondary to a failure to expand.

PDX-1 has been reported to regulate the expansion of pancreatic endocrine cells during development, and PDX-1 has been considered a regulator of β cell-specific genes and proteins in the mature p cell. These observations provide direct evidence that, in addition to its role in β cell-specific gene regulation, PDX-1 interacts with the insulin signaling system and is a critical regulator of p cell plasticity for the maintenance of β cell populations and glucose homeostasis.

Treatment of Diabetes Mellitus

The methods described herein are useful in treating disorders associated with a loss of insulin-secretin p cells, e.g., diabetes mellitus (DM). The methods can include administering a modulator of E-cadherin/β-catenin signalling to the subject. The modulators can be administered systemically or locally, e.g., by injection or implantation of a device that provides a steady dose of the modulator to the pancreatic tissues, e.g., to the islets. Such devices are known in the art, and include micro-pumps and controlled-release matrices, e.g., matrices that break down over time, releasing the modulator into the tissue.

Alternatively, the methods include cell-based therapies. For example, The methods can include implanting into a subject a population of re-differentiated β cells that has been expanded or increased by a method described herein. In some embodiments, the cells are autologous, e.g., they come from the same subject into which they will be transplanted. Surgical methods for implanting such cells are known in the art, and include minimally-invasive, endoscopic methods. Generally, for humans, it is desirable to implant at least about a mean (±SD) islet mass of 10,000 islet equivalents per kilogram of body weight, see, e.g., Shapiro et al., N. Engl. J. Med. 343(4):230-8 (2000). In some embodiments, the cells are substantially fully re-differentiated. In some embodiments, the cells are not fully re-differentiated.

Screening Assays

In part, the methods described herein include assays for the identification and verification of compounds that can cause fully differentiated, insulin secreting β cells to de-differentiate. Such compounds can be identified from information that may be available in the art, or using laboratory methods for identifying modulators, i.e., test compounds or agents (e.g., proteins, peptides, peptidomimetics, peptoids, small inorganic molecules, small non-nucleic acid organic molecules, nucleic acids (e.g., anti-sense nucleic acids, siRNA, oligonucleotides, synthetic oligonucleotides), or other drugs) that modulate E-cadherin/β-catenin signalling. For example, such a compound may bind to E-cadherin and have an inhibitory effect on E-cadherin signalling, or cause β-catenin to translocate to the nucleus, e.g., to be sequestered in the nucleus. Compounds thus identified can be used to increase populations of β-cells, e.g., in a method described herein.

In some cases, an assay involves the identification of a compound that modulates E-cadherin/β-catenin signalling, and determining whether the compound can increase β-cell populations, e.g., in vitro or in vivo. Methods of identifying a compound that modulates E-cadherin/β-catenin signalling are known in the art and described herein. Compounds previously identified as able to modulate E-cadherin/β-catenin signalling can also be used in the methods described herein.

In some embodiments, an assay for verifying that a compound is suitable for use in a method described herein is a cell-based assay in which a fully differentiated β cell or population of such cells is contacted with a test compound that modulates E-cadherin/β-catenin signalling, and the ability of the test compound to induce proliferation is determined. Those test compounds that are demonstrated to induce proliferation can be further evaluated by removing the test compound from the cells, and determining whether the cells re-differentiate to become glucose-sensitive insulin secreting β cells.

Test compounds that can be used in the methods described herein can include those obtained using any of the numerous approaches in combinatorial library methods known in the art, including: biological libraries (e.g., peptides, polypeptides, or nucleic acids); peptoid libraries (libraries of molecules having the functionalities of peptides, but with a novel, non-peptide backbone which are resistant to enzymatic degradation but which nevertheless remain bioactive; e.g., Zuckermann et al., J. Med. Chem., 37:2678-2685 (1994)); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer, or small molecule libraries of compounds (Lam, Anticancer Drug Des. 12:145 (1997)). As used herein, “small molecules” refers to small organic or inorganic molecules of molecular weight below about 5,000 Daltons.

Examples of methods for the synthesis of molecular libraries can be found in the art, for example in: DeWitt et al., Proc. Natl. Acad. Sci. USA, 90:6909 (1993); Erb et al., Proc. Natl. Acad. Sci. USA, 91:11422 (1994); Zuckermann et al., J. Med. Chem., 37:2678 (1994); Cho et al., Science, 261:1303 (1993); Carrell et al., Angew. Chem. Int. Ed. Engl., 33:2059 (1994); Carell et al., Angew. Chem. Int. Ed. Engl., 33:2061 (1994); and in Gallop et al., J. Med. Chem., 37:1233 (1994). Libraries of compounds can be presented in solution (e.g., Houghten, Biotechniques, 13:412-421 (1992)), or on beads (Lam, Nature, 354:82-84 (1991)), chips (Fodor, Nature, 364:555-556 (1993)), bacteria (Ladner, U.S. Pat. No. 5,223,409), spores (Ladner, U.S. Pat. No. 5,223,409), plasmids (Cull et al., Proc. Natl. Acad. Sci. USA, 89:1865-1869 (1992)), or on phage (Scott and Smith, Science, 249:386-390 (1990); Devlin, Science, 249:404-406 (1990); Cwirla et al., Proc. Natl. Acad. Sci. USA, 87:6378-6382 (1990); Felici, J. Mol. Biol., 222:301-310 (1991); Ladner supra).

A test compound that has been screened by a method described herein and determined to be suitable, can be considered a candidate compound. A candidate compound that has been screened, e.g., in an in vivo model of a disorder, e.g., diabetes, and determined to have a desirable effect on the disorder, e.g., on one or more symptoms of the disorder, can be considered a candidate therapeutic agent. Candidate therapeutic agents, once screened in a clinical setting, are therapeutic agents. Candidate therapeutic agents and therapeutic agents can be optionally optimized and/or derivatized, and formulated with physiologically acceptable carriers and/or excipients to form pharmaceutical compositions.

The invention is further described in the following examples, which do not limit the scope of the invention described in the claims.

### EXAMPLES

### Example 1

### Growth and Development of IR/IRS-1/PDX-1 Triple Heterozygous Knockout Mice

This Example describes the generation and maintenance of IR/IRS-1/PDX-1 triple heterozygous knockout (KO) mice.

IR/IRS-1 mice, PDX-1 heterozygotes, and LIRKOs were maintained on the original mixed background (C57BL/6×129Sv×DBA/2) and bred at the Brandeis Animal Facility (Brandeis University, Waltham, Mass., USA) on a 12-hour light/12-hour dark cycle with ad lib water and food (Mouse Diet 9F; PMI Nutrition International). A mixed mating scheme was adopted for breeding the two groups of mice. In the first group, both males and females carrying either the IR, IRS-1, or PDX-1 alleles were used. In the second group, males and females which were homozygous for loxP sites (IRLox) were mated with mice expressing Cre recombinase on an albumin promoter. The breeding generated several genotypes for the 2 studies. The genotypes of mice in the IR/IRS-1/PDX-1 study included the WT, IR, IRS-1, IR/IRS-1, PDX-1, IRS-1/PDX-1, IR/PDX-1, and TKO groups. In the LIRKO/PDX-1 study, the genotypes included mice homozygous for loxP sites (IRLox), PDX-1 mice, LIRKO mice, and LIRKO/PDX-1 mice. Genotyping was performed by PCR analysis of genomic DNA obtained from tail snips (Kulkarni et al., 1999. Cell. 96:329-339). All procedures were approved by the Joslin Diabetes Center Institutional Animal Care and Use Committee and performed in accordance with its guidelines.

In the Examples described herein, statistical analysis was performed by the Student's t test or ANOVA as appropriate, and values were considered significant at P<0.05.

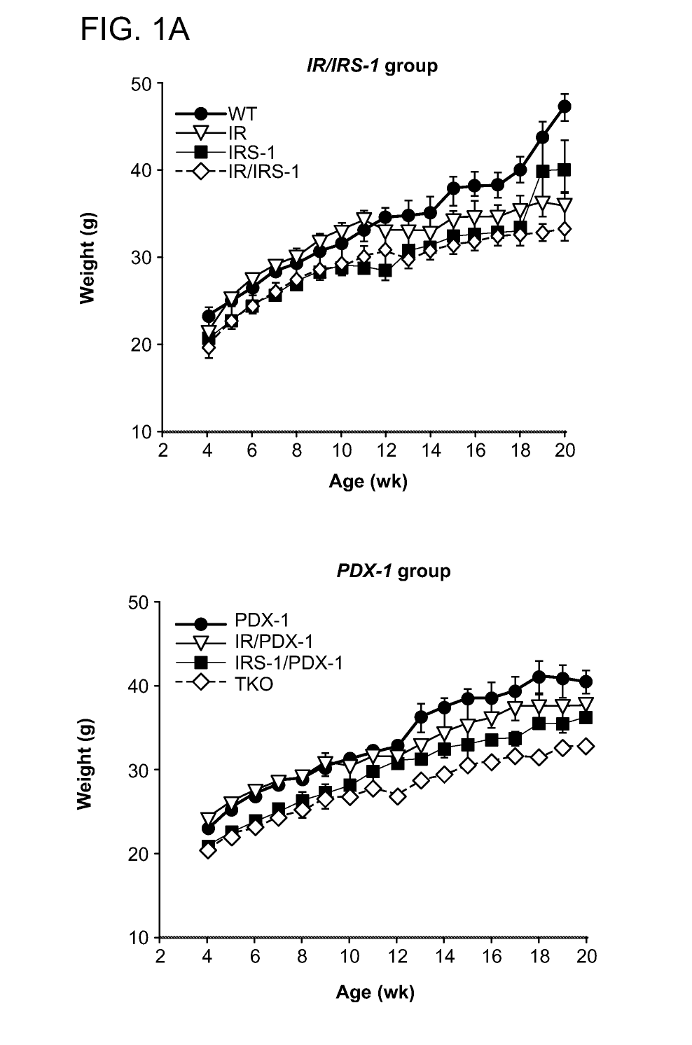

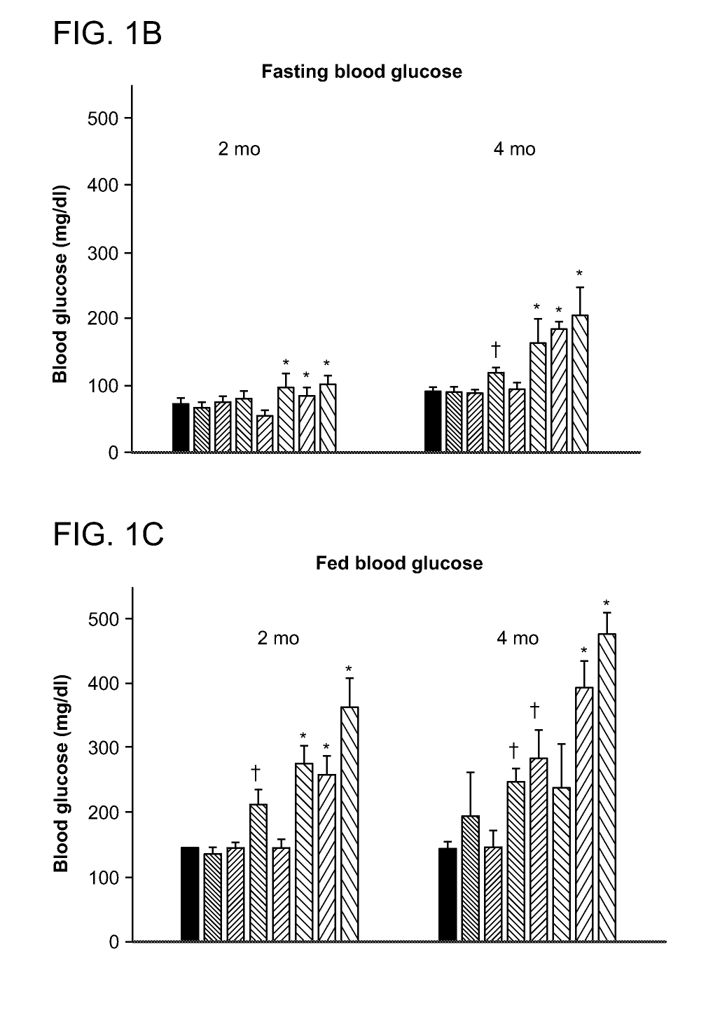

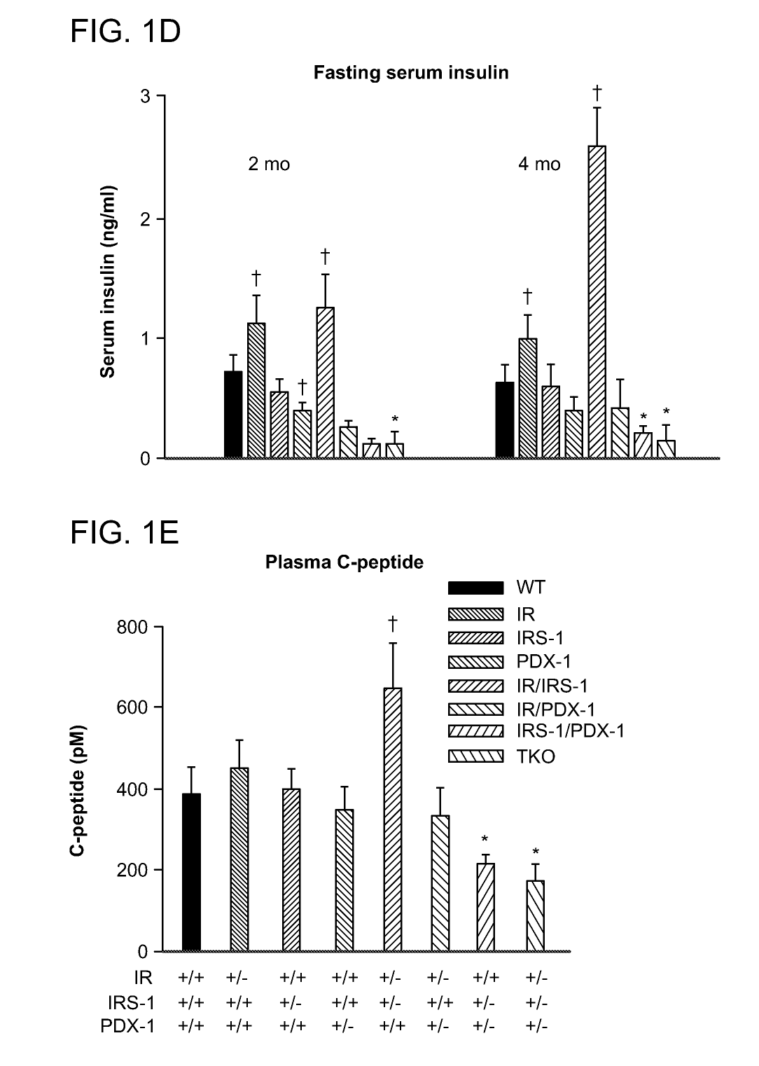

All mice were born in a normal mendelian ratio in the different groups, and no embryonic lethality was observed. Mice that were heterozygous for IRS-1 —the IRS-1 heterozygous (IRS-1), IR/IRS-1 double heterozygous (IR/IRS-1), IRS-1/PDX-1 double heterozygous (IRS-1/PDX-1), and IR/IRS-1/PDX-1 triple heterozygous KO (TKO) groups—exhibited a significant (10-15%) reduction in body weight (FIG. 1A, left and right panels), consistent with earlier reports (Kulkarni et al., 1999. J. Clin. Invest. 104:R69-R75, Araki et al., 1994. Nature. 372:186-190). The further decrease in body weights in TKO and IRS-1/PDX-1 males and females compared with the IRS-1 and ER/IRS-1 groups is likely caused by glycosuria and wasting due to severe diabetes in older mice. Qualitatively similar but milder phenotypes were observed in females. Therefore, data from males only will be presented herein.

### Example 2

### TKO Mice Manifest Severe Hyperglycemia, Hypoinsulinemia, Loss of Acute Phase Insulin Secretion, and Glucose Intolerance

Hyperglycemia, hypoinsulinemia, acute phase insulin secretion, and glucose tolerance were evaluated in the mice described in Example 1.

Overnight fasting (14 hours) and fed glucose levels were measured by a glucometer (Elite; Bayer Corp.) using tail vein blood. Serum insulin levels were assayed by ELISA using mouse insulin standards (Crystal Chem. Inc.). C-peptide was measured by an RIA kit (Linco Research Inc.). Pancreatic insulin content was measured in acid-ethanol extracts of homogenized pancreas as described previously (Kulkarni et al., 1999. Cell. 96:329-339). Glucose and insulin tolerance tests and acute phase insulin release experiments were performed essentially as described previously (Kulkarni et al., 1999. Cell. 96:329-339).

IR/IRS-1 mice showed normal glucose levels at 2 months that worsened slightly at 4 months of age, especially in the fed states. In PDX-1 heterozygotes, however, the fed blood glucose was mildly elevated at 2 months, and the hyperglycemia persisted at 4 months. At this time IR and IR/IRS-1 mice showed hyperinsulinemia (Bruning et al., 1997. Cell. 88:561-572), and this was adequate to maintain normoglycemia. By age 4 months, the double heterozygous mice showed hyperglycemia despite the presence of even higher levels of circulating insulin (FIGS. 1, B-D). In mice with combined heterozygosity for PDX-1, IR, and/or IRS-1, significantly higher blood glucose levels were detected in the fasted and fed states by 2 months, and the hyperglycemia worsened by 4 months in all groups, except IR/PDX-1 compared with PDX-1 single heterozygotes (FIGS. 1, B and C). The hyperglycemia in IRS-1/PDX-1 and TKO compound KOs could be attributed to significantly lower serum insulin levels compared with those in individual heterozygotes and controls (FIG. 1D). Plasma C-peptide, measured at 3 months of age, showed a similar trend, suggesting a significantly reduced insulin output from the endocrine pancreas (FIG. 1E).

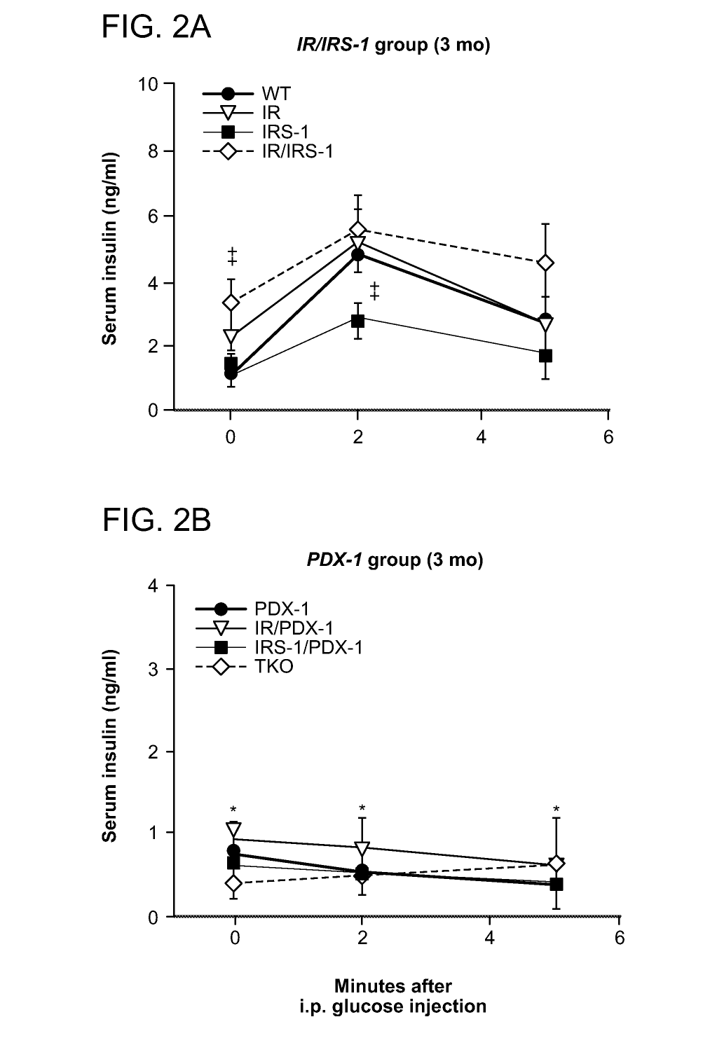

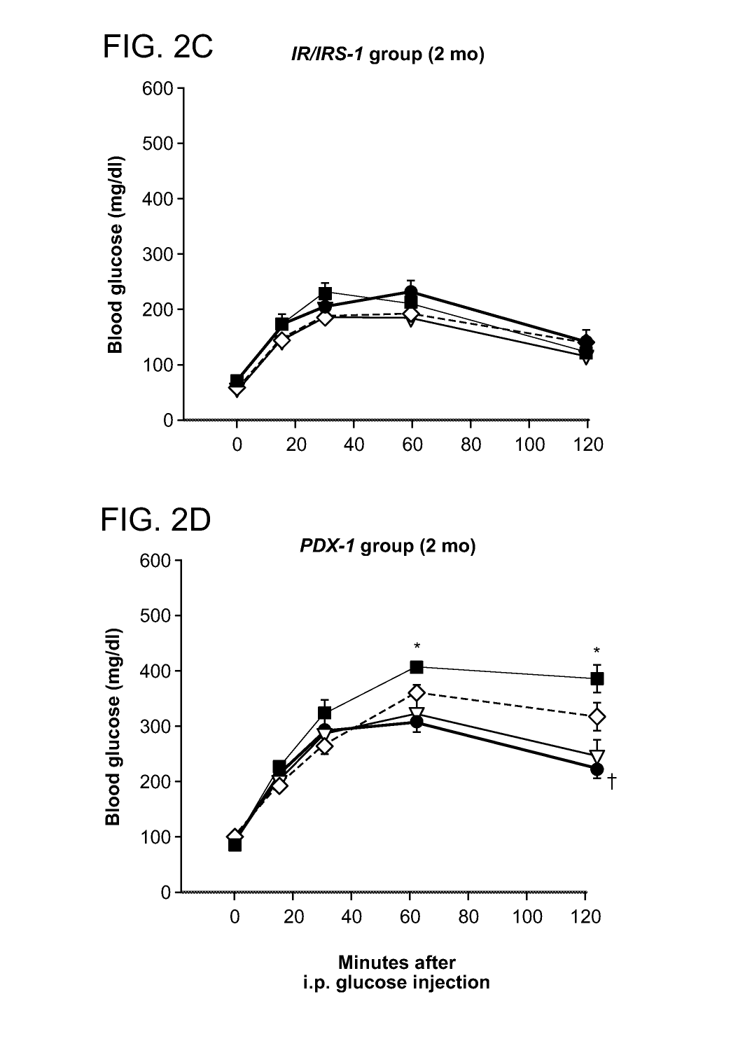

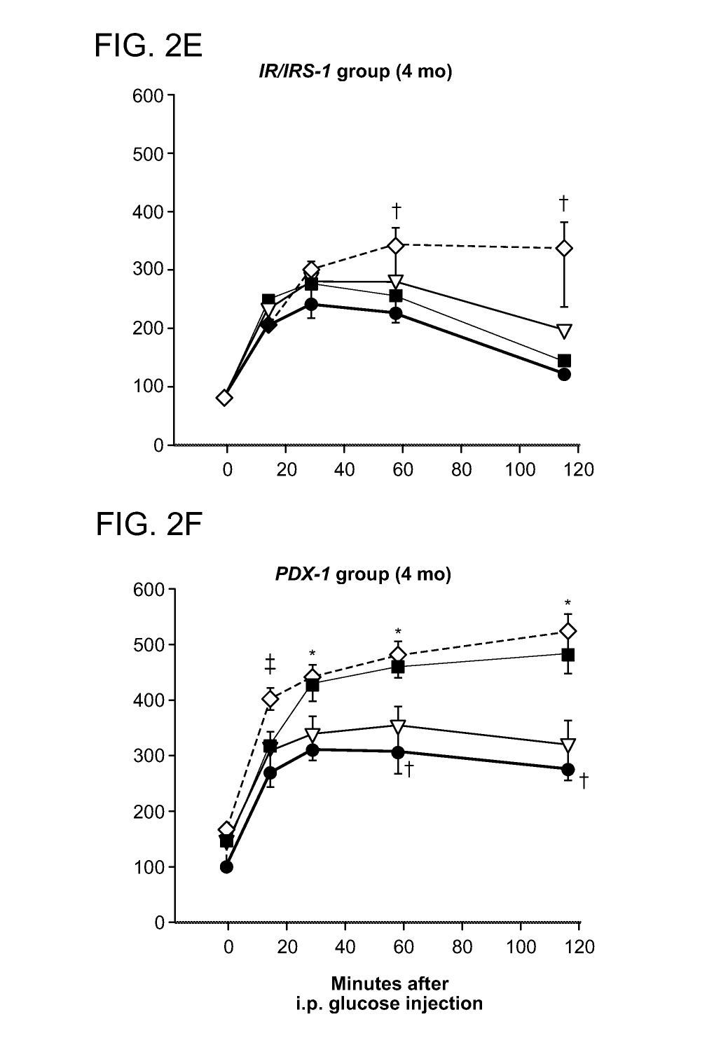

Insulin/IGF-I signaling in β cells has been demonstrated to play a role in glucose-stimulated insulin secretion (Kulkarni, 2002. Biochem. Soc. Trans. 30:317-322; Kulkarni et al., 2002. Nat. Genet. 31:111-115; Xuan et al., 2002. J. Clin. Invest. 110:1011-1019), while PDX-1 is known to regulate expression of Glut2 and glucokinase genes (Jonsson et al., 1994. Nature. 371:606-609; Waeber et al., 1996. Mol. Endocrinol. 10:1327-1334). Furthermore, insulin/IGF-I signaling in the islet has been linked with glucokinase (Otani et al., 2004. Am. J. Physiol. Endocrinol. Metab. 286:E41-E49; Kulkarni et al., 2002. Nat. Genet. 31:111-115; Leibiger et al., 2001. Mol. Cell. 7:559-570; Da Silva et al., 2004. Biochem. J. 377:149-158) and PDX-1 expression (Da Silva et al., 2004. Biochem. J. 377:149-158). To evaluate insulin secretory function, mice were injected with glucose by the intraperitoneal route. An approximately 3-fold secretory response was observed in the WT, while virtually no responses were observed in all the groups that were heterozygous for PDX-1, consistent with an earlier study (FIGS. 2, A and B) (Shih et al., 2002. Proc. Natl. Acad. Sci. U.S.A. 99:3818-3823). Consequently, the groups heterozygous for PDX-1 displayed glucose intolerance by 2 months, and the glucose levels were highest at all time points during the glucose tolerance test in IRS-1/PDX-1 and TKO mice (FIG. 2D), compared with respective controls (FIG. 2C), and continued to worsen as the mice aged (FIGS. 2, E and F). Mild glucose intolerance was observed in the PDX-1 group (FIG. 2D), while the IR/IRS-1 mice were glucose intolerant only at age 4 months (FIG. 2E). No significant differences in insulin sensitivity, as measured by an insulin tolerance test, were observed between the groups at 2 months (data not shown). The severe hyperglycemia in the IRS-1/PDX-1 and TKO groups is likely due to relatively low circulating insulin levels in the compound KOs.

### Example 3

### Altered PDX-1 Protein Expression and Reduced β Cell Mass in TKO Islets

PDX-1 protein expression and β cell mass were evaluated in TKO islets.

β Cell Mass and Immunohistochemistry:

Mice were anesthetized, and pancreata were rapidly dissected, weighed, fixed in Bouin's or 4% paraformaldehyde solution, embedded in paraffin, sectioned, and stained as described below. β Cell mass was estimated by morphometric analysis as described previously (Kulkarni et al., 1999. Cell. 96:329-339, Michael et al., 2000. Mol. Cell. 6:87-97). Five-micrometer sections of paraffin-embedded pancreas were dewaxed using xylene, rehydrated through serial dilutions of ethyl alcohol, and subjected to antigen retrieval using 10 mM citrate (pH 6.1) or DAKO High pH antigen-retrieval solution (DAKO Corp.). The sections were washed and stained with the respective antibodies in staining buffer with 100 mM NaCl, 3% BSA, 1% Triton X-100, and 50 mM NaPO₄ (pH 7.4). Primary antibodies included guinea pig anti-insulin (Linco Research Inc.) or sheep anti-insulin (The Binding Site Ltd.), rabbit anti-somatostatin (DAKO), mouse anti-glucagon (Sigma-Aldrich), rabbit anti-Glut2 (a gift from B. Thorens, University of Lausanne, Lausanne, Switzerland), rabbit anti-synaptophysin (DAKO), mouse anti-PCNA (DAKO), β-catenin (BD Biosciences/Pharmingen), E-cadherin (Valeant Pharmaceuticals), rabbit anti-caspase-3, and Texas red-conjugated lectin (EY Laboratories Inc.). The secondary antibodies used included Cy2 and Cy3 fluorescent conjugated dyes (Jackson ImmunoResearch Laboratories Inc.). Sections were viewed and photographed using a DeltaVision deconvolution microscope (Applied Precision LLC). For immunohistochemistry, sections were stained with primary antibodies (DAKO Corp.), followed by incubation with secondary antibodies (Envision Plus; DAKO Corp.). For color development, diaminobenzidine (DAB) chromogen (DAKO) was used and counterstained with Mayer's hematoxylin (diluted 1:1). Several chromogens were used for counterstaining as follows: for PCNA and caspase-3, DAB hematoxylin counterstaining was used; for PCNA and β-catenin, grey-black immunoprecipitates (Vector SG; Vector Laboratories Inc.) were used to visualize β-catenin and VIP substrate (red-purple color; Vector Laboratories Inc.) was used to visualize PCNA; for β-catenin and E-cadherin immunostaining, β-catenin was visualized with DAB (brown color) and E-cadherin with brown-black immunoprecipitates (Vector SG; Vector Laboratories Inc.); Nuclear Red (DAKO Corp.) was used for counterstaining of nuclei.

Western Blotting:

Whole pancreata were removed from mice and promptly homogenized using a polytron in 5 ml of 1% SDS/6 M urea lysis buffer containing protease and phosphatase inhibitors. Islets were homogenized using a hand-held homogenizer in the SDS-urea lysis buffer and processed similarly to whole pancreata. Equal proteins from samples (assessed by Micro BCA protocols; Pierce Biotechnology Inc.) were resolved by 10% SDS-PAGE, and the gel was transferred to nitrocellulose and probed for PDX-1 protein with anti-PDX-1 antibody (a gift from J. Habener, Massachusetts General Hospital, Boston, Mass., USA) or anti-CREB antibodies using ECL (Amersham Biosciences).

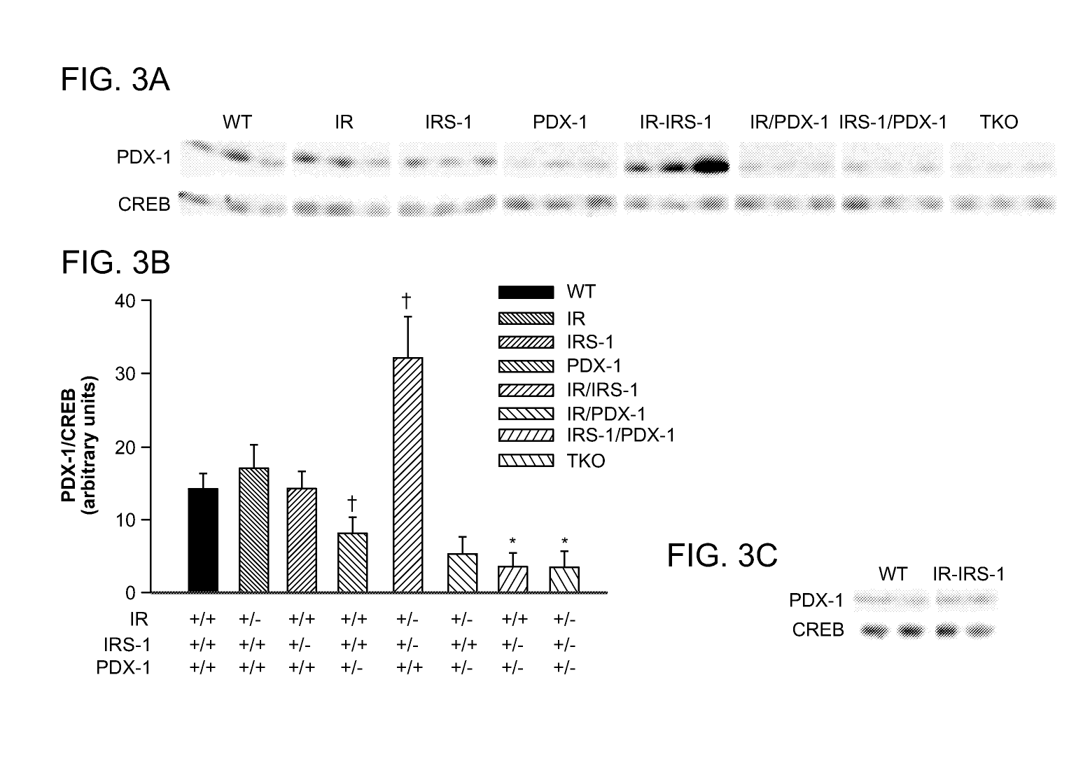

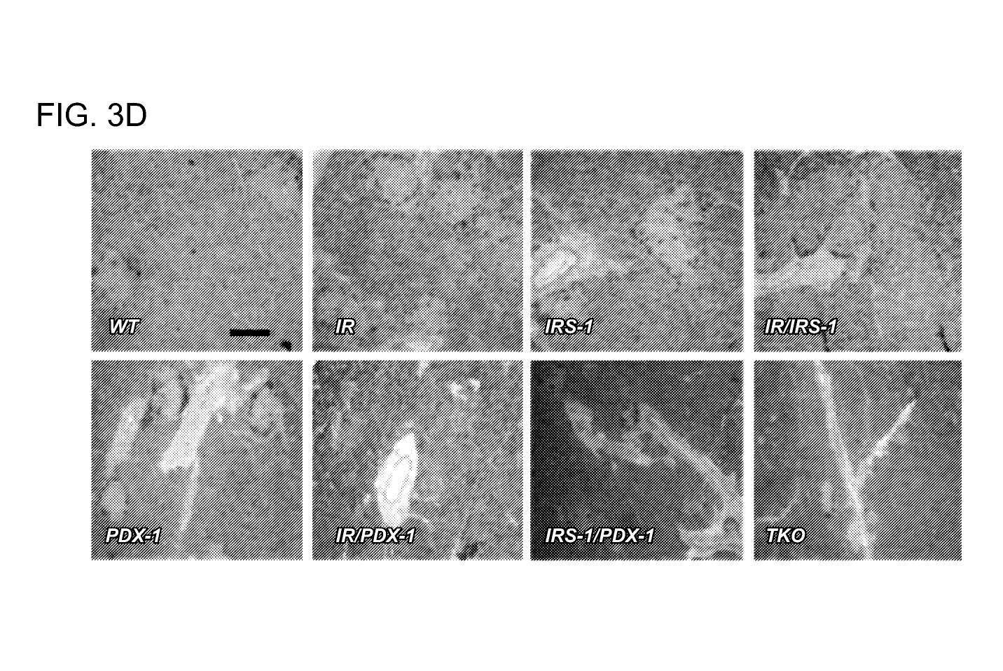

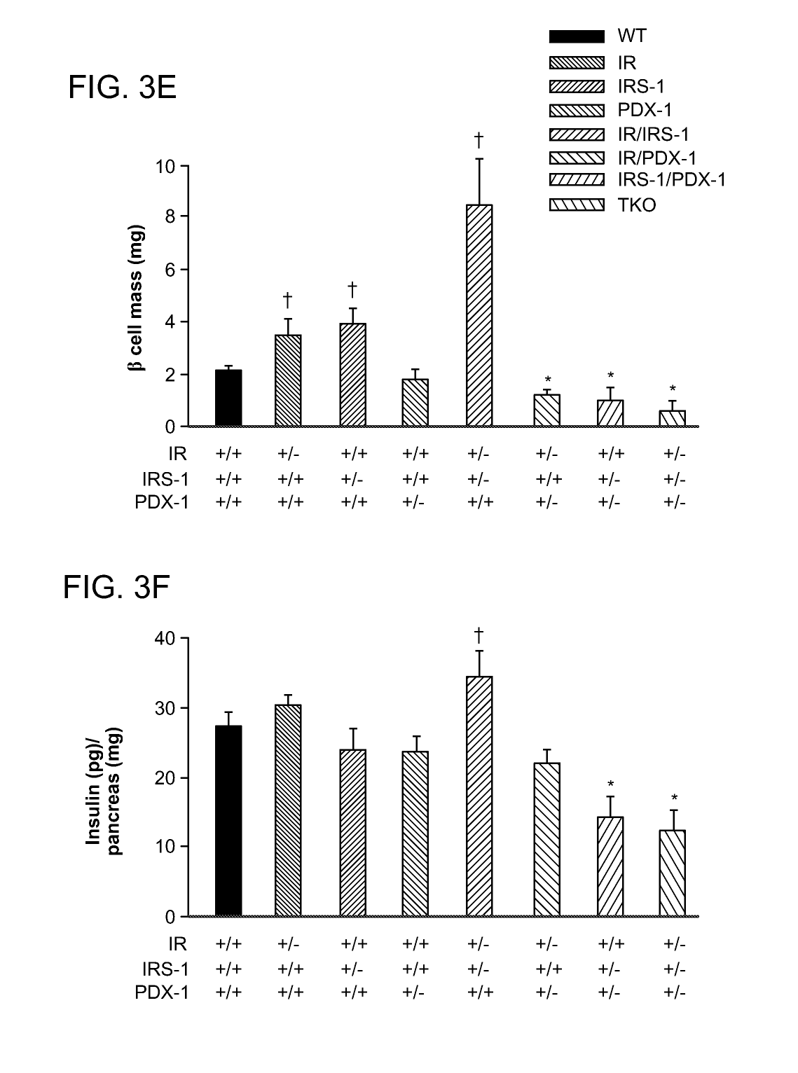

Western blotting of proteins from whole pancreas, to quantify the partial loss of PDX-1, showed approximately 50% lower levels of PDX-1 protein in PDX-1 heterozygotes compared with the WT, IR, and IRS-1 groups (FIGS. 3, A and B). However, the PDX-1 levels were reduced even further in the IRS-1/PDX-1 and TKO groups compared with the PDX-1 group; in fact, PDX-1 was almost undetectable in these groups. By contrast, a 2.3-fold higher level of PDX-1 protein was observed in IR/IRS-1 mice compared with WT mice (FIG. 3B). When PDX-1 was expressed per milligram islet protein, however, the values were not significantly different from those for the WT group, which suggests that the increase in PDX-1 protein levels in the pancreas in IR/IRS-1 mice is due to islet hyperplasia (FIG. 3C).

Morphometric analysis of β cell mass at 3 months of age showed a 2- to 4-fold increase in the IR/IRS-1 group (and about a 9-fold increase at 6 months; data not shown), compared with the WT (FIGS. 3, D and E) (Bruning et al., 1997. Cell. 88:561-572; Kulkarni et al., 2003. Diabetes. 52:1528-1534). Similarly, IR and IRS-1 heterozygous mice showed a significant increase in β cell mass, which indicates a compensatory response of islets to insulin resistance (Kulkarni et al., 1999. J. Clin. Invest. 104:R69-R75; Kubota et al., 2000. Diabetes. 49:1880-1889; Kido et al., 2000. J. Clin. Invest. 105:199-205). In contrast, a milder, statistically insignificant reduction in β cell mass was detected in PDX-1 heterozygotes (Dutta et al., 1998. Nature. 392:560). When PDX-1 heterozygosity was combined with heterozygosity for 1 or more of the insulin signaling proteins (IR/PDX-1, IRS-1/PDX-1, and TKO), a dramatic decrease in β cell mass was observed compared with that in PDX-1 heterozygotes alone (FIG. 3E). In fact, few or no islets were detected in pancreas sections in a majority of IRS-1/PDX-1 and TKO mice. The reduced β cell mass was consistent with reduced pancreatic insulin content in the various groups, the lowest content being observed in the IRS-1/PDX-1 and TKO groups (FIG. 3F). No significant differences were detected in non-β cell mass among the groups.

### Example 4

### Decreased Expression of PDX-1 Targets in TKO Mice

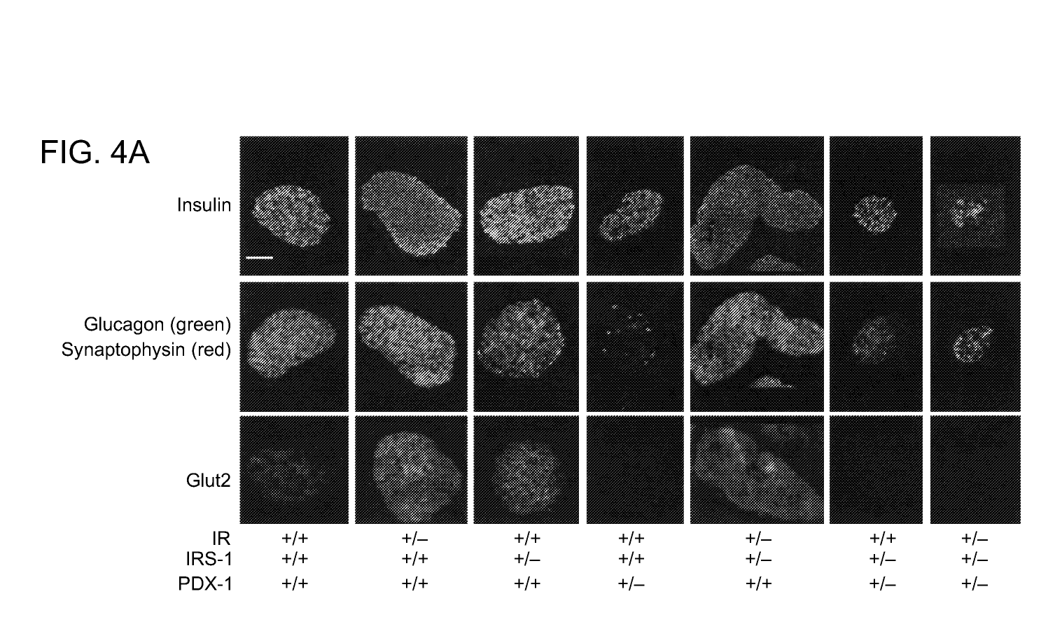

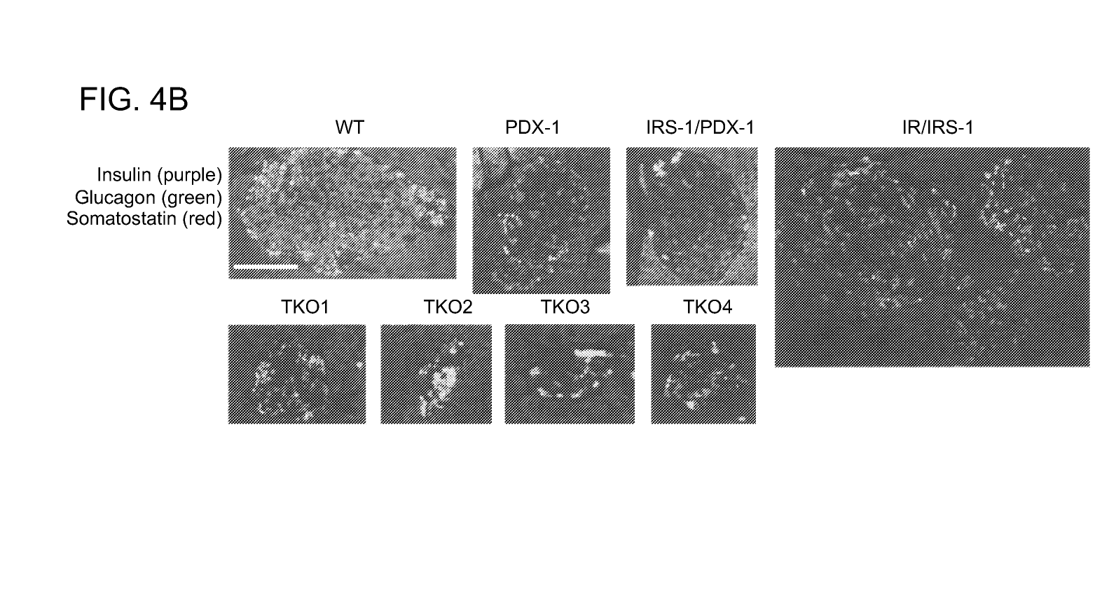

Pancreata from the various groups were evaluated for expression of key markers of islet function including insulin (FIG. 4A, upper panels) and glucagon (FIG. 4A, green, middle panels). Insulin- and glucagon-positive cells could be detected in all genotypes, which indicates that differentiation of α and β cells was not disrupted in the compound KOs. As expected, IR/IRS-1 mice showed an approximately 3-fold increase in islet size, due to an increase in β cell mass, with the non-β cells scattered throughout the islets (FIG. 4A, upper and middle panels) (13, 28). To examine whether the islet hyperplasia was due to an increase in non-β cell types in the islets, pancreas sections from IR/IRS-1 and PDX-1-deficient groups were costained for expression of insulin, glucagon, and somatostatin (FIG. 4B). The hyperplastic islets in IR/IRS-1 mice were characterized by an increase in β cells with glucagon- and somatostatin-positive cells scattered within the islet. Consistent with a lack of significant difference in circulating glucagon levels among groups, the number of glucagon- and somatostatin-positive cells appeared to be in similar proportions in all groups. Pancreas sections from IRS-1/PDX-1 and TKO mice showed very small islets with a reduced number of β cells, which resulted in the appearance of an increase in glucagon-staining α cells within the core of the islet (FIG. 4A, middle panels). The absence of a significant alteration in non-β cell mass and in circulating glucagon levels (data not shown) in the compound KOs suggested a predominant effect on β cells. Furthermore, no differences in circulating levels of total glucagon-like peptide-1 were detected between groups, which suggests that the altered β cell mass is unlikely to be linked with glucagon-like peptide-1 action (WT, 14±4; IR/IRS-1, 16±6; PDX-1, 15±7; IRS-1/PDX-1, 22±10; TKO, 17±4 pmol/l; n=3-5, P=NS).

Islets were also examined for alterations in Glut2, a key target for PDX-1 (24). Robust Glut2 expression was detected in IR, IRS-1, and IR/IRS-1 double heterozygote islets (FIG. 4A, lower panels). However, the protein was virtually undetectable in all mice carrying a single allele for PDX-1, including PDX-1, IRS-1/PDX-1, and TKO mice. These observations are consistent with earlier observations that Glut2 is a direct transcriptional target for PDX-1 (24). In addition, based on preliminary gene expression profiling studies in islets from PDX-1 heterozygotes (M. Montminy, U.S. Jhala, and J. Kushner, unpublished observations), expression of synaptophysin, another PDX-1 target and an integral component of the neuroendocrine secretory granule, was also examined (FIG. 4A, red, middle panels). The expression pattern of synaptophysin paralleled that of Glut2 (FIG. 4A, lower panels). Thus, in addition to regulating insulin, PDX-1 also regulates other genes that are important for glucose sensing and for insulin exocytosis, which may in part explain the loss of acute phase insulin secretion observed in the PDX-1-deficient mice.

### Example 5

### Increased β Cell Apoptosis and Diminished β Cell Replication in TKO Islets

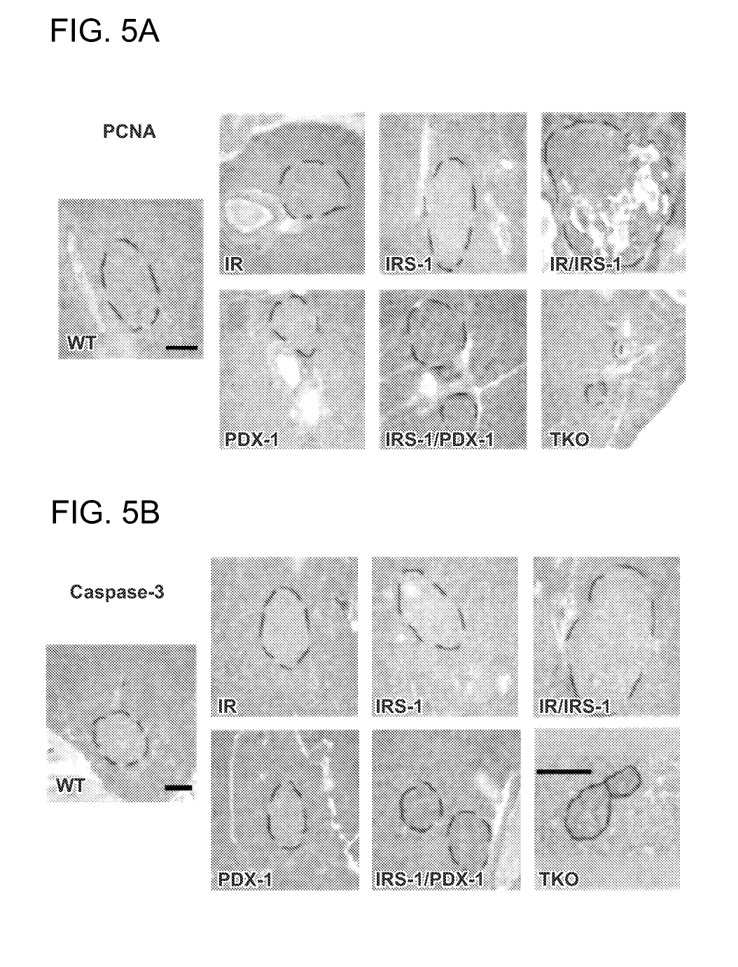

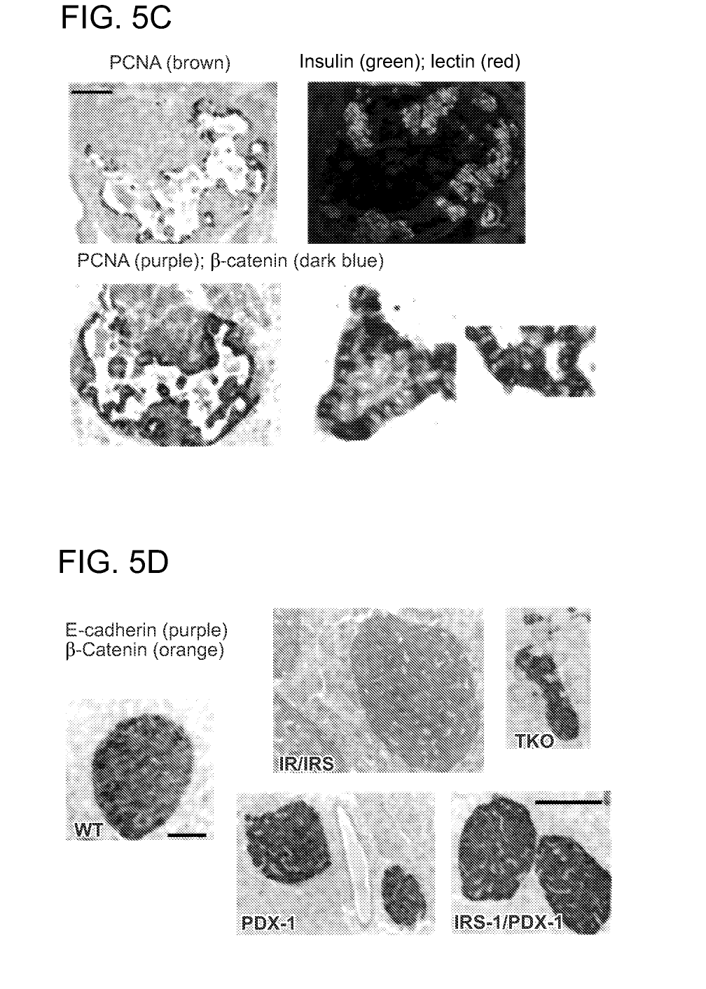

β Cell mass in the adult mouse is maintained by a balance between newly generated β cells (by β cell replication and neogenesis from potential ductal precursors) and β cell death by apoptosis or necrosis (Dor et al., 2004. Nature. 429:41-46; Bonner-Weir, 2000. Endocrinology. 141:1926-1929). To obtain an estimate of these parameters and to isolate the potential mechanism underlying the poor islet compensation in the IRS-1/PDX-1 and TKO groups, the pancreas was examined for markers of proliferation and apoptosis in these mice (Kelman, 1997. Oncogene. 14:629-640; Budihardjo et al., 1999. Annu. Rev. Cell Dev. Biol. 15:269-290). In double heterozygous mice carrying both copies of PDX-1, a striking pattern and increase in proliferating cellular nuclear antigen (PCNA) staining in cells was observed within the islets, while virtually no PCNA+ cells were evident in other groups (FIG. 5A; Table 1). Furthermore, the PCNA+ cells showed a distinct morphology compared with surrounding β cells. In contrast, examination of the pancreas for activated caspase-3, a marker for the end stage of apoptosis, showed increased staining in islets of TKO mice. Some caspase-3+ cells were also observed in islets of IRS-1/PDX-1 mice, while few or none could be detected in other groups (FIG. 5B; Table 1). Thus, the islet hyperplasia in ER/IRS-1 mice is predominantly caused by a robust expansion of β cells, whereas the lack of compensatory response in IRS-1/PDX-1 and TKO groups is most likely a result of the combined absence of β cell proliferation and increased β cell apoptosis.

TABLE 1
Reduced PCNA+ cells and increased Caspase 3+ cells
in TKO islets.

| | | | | | IR/ | IRS-1/ | |
|---|---|---|---|---|---|---|---|
| | WT | IR | IRS-1 | PDX-1 | IRS-1 | PDX-1 | TKO |
| PCNA-Positive | 0.5 | 0.3 | 0.3 | 0.6 | 28 | 4 | 0.1 |
| cells/islet | | | | | | | |
| Caspase-3- | 1.3 | 2.2 | 1.8 | 3.1 | 4.1 | 11.2 | 26.4 |
| Positive | | | | | | | |
| cells/islet | | | | | | | |

Legend to Table 1. PCNA-positive cells and caspase 3-positive cells in islets in compound knockout mice. At least 30 islets from 2 different mice were examined for each genotype. Average of data from at least 2 different animals in each group are shown.

To evaluate the origin of the PCNA+ cells, serial sections of pancreas were stained with a fluorescence-labeled duct-specific lectin (DBA) (Kobayashi et al., 2002. Biochem. Biophys. Res. Commun. 293:691-697). Ductal epithelial cells showed the expected positive staining; however, none of the cells in the area of proliferation were positive for DBA-lectin immunohistochemistry for duct-specific glycoconjugates (FIG. 5C). These data strongly suggest that the proliferating cells, rather than being of ductal origin, are likely replicating β cells with metaplastic changes. Similar metaplastic changes in rapidly expanding epithelial cell types are associated with marked changes in adherens junctions between cells (Potter et al., 1999. Endocr. Rev. 20:207-239). In the pancreas, the cadherin-catenin complex regulates aggregation of β cells in vivo (Dahl et al., H. 1996. Development. 122:2895-2902) and participates in paracrine signal transmission from neighboring cells by interacting with growth factor receptors (Williams et al., 1994. Neuron. 13:583-594; Lopez et al., T., and Hanahan, D. 2002. Cancer Cell. 1:339-353). Therefore, the islets were examined for expression of 2 important components of the adherens junction, namely E-cadherin, a transmembrane protein that mediates cell-to-cell association, and β-catenin, which anchors E-cadherin to the cytoskeletal network in the cells (Potter et al., 1999. Endocr. Rev. 20:207-239; Dahl et al., H.1996. Development. 122:2895-2902). β Cells in WT islets stained positive for both E-cadherin and membrane-bound β-catenin, indicating strong cell-to-cell adhesion, which normally occurs in well-clustered islets (FIG. 5D). E-cadherin, however, was dramatically downregulated and undetectable in the hyperplastic islets in IR/IRS-1 mice (FIG. 5D). In marked contrast, PDX-1 heterozygotes strongly expressed E-cadherin but showed a virtual absence of β-catenin, suggesting a fundamental alteration in adherens junctions in 3 cells of the PDX-1 islets (FIG. 5D).

These data are reminiscent of changes in adhesion protein expression that are normally observed in rapidly proliferating cells, especially during tumorigenesis (Potter et al., 1999. Endocr. Rev. 20:207-239). In such a model, the loss of E-cadherin leads to a dissolution of the adherens junction and allows the membrane-bound β-catenin to migrate into the cytoplasm and nucleus and participate in activation of genes required for proliferation (Potter et al., 1999. Endocr. Rev. 20:207-239). Accordingly, the PCNA+ cells in the hyperplastic islets from IR/IRS-1 mice showed a decrease in membrane-bound β-catenin and an increase in translocation into the cytoplasm and nucleus. In the surrounding nonproliferating β cells, however, β-catenin was found to be membrane bound. The colocalization of PCNA and β-catenin in the nucleus strongly suggests the involvement of β-catenin in active proliferation (FIG. 5C, lower right panels). On the other hand, the morphological changes and downregulation of E-cadherin was not observed in TKO or IRS-1/PDX-1 islets, which suggests a limited ability of the mutant β cells to respond to the insulin resistance in the presence of PDX-1 haploinsufficiency. Surprisingly, β cells in TKO islets stained positive for caspase-3, suggesting a loss of β cells due to apoptosis (FIG. 5B).

### Example 6

### PDX-1 Haploinsufficiency Limits Islet Hyperplasia in a Second Model of Insulin Resistance, the LIRKO

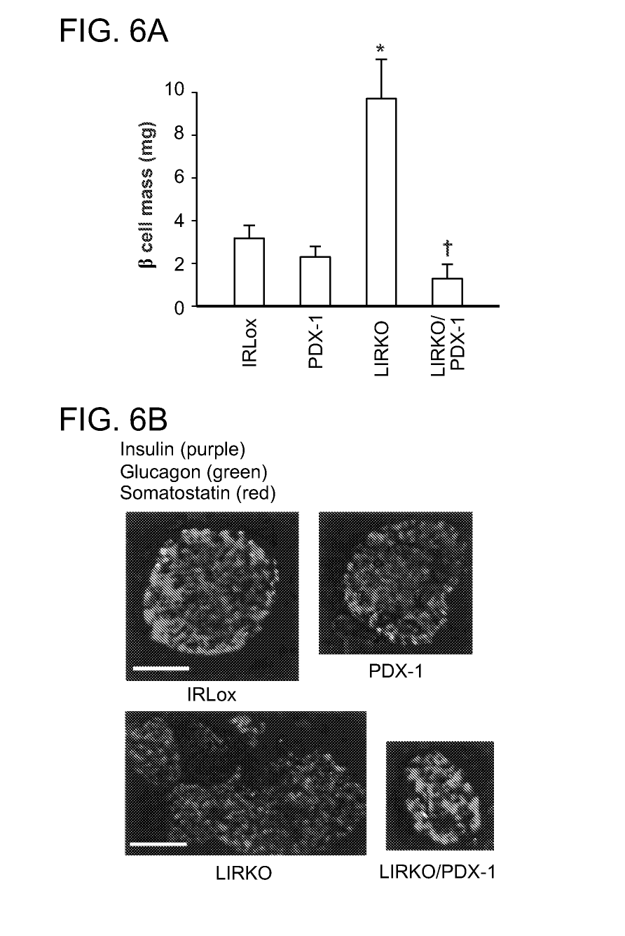

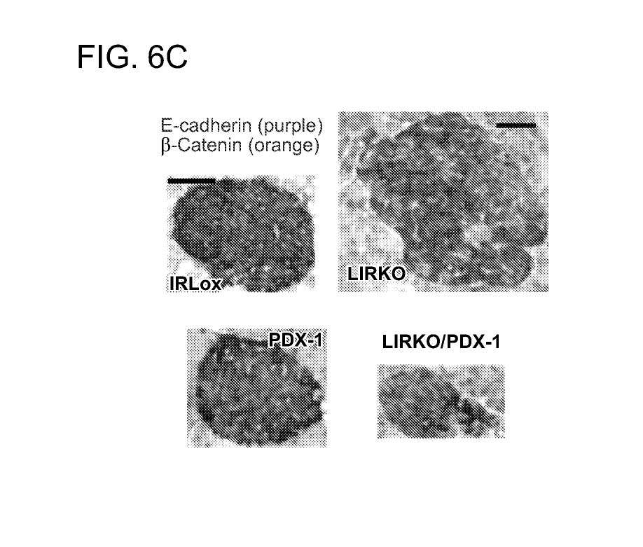

The next series of studies examined whether PDX-1 haploinsufficiency can also limit the islet hyperplastic response in other models of insulin resistance. The role of PDX-1 was examined in the LIRKO mouse, which also shows severe hyperinsulinemia and robust islet hyperplasia (Michael et al., 2000. Mol. Cell. 6:87-97). Compound KOs were generated by crossing PDX-1 heterozygotes with mice bearing a homozygous deletion of exon 4 of the IR gene. This model of insulin resistance has been described earlier (Michael et al., 2000. Mol. Cell. 6:87-97). Again, an approximately 4-fold increase in β cell mass was observed in LIRKOs, while in contrast, in LIRKO/PDX-1 compound KOs, the hyperplastic response was virtually absent and instead the islets were small with non-β cells scattered throughout the islet (FIGS. 6A and 6B). Thus, haploinsufficiency of PDX-1 limited the ability of islets to compensate in a second model, suggesting that the homeodomain protein is a crucial component in the β cell proliferative response to insulin resistance. As in the IR/IRS-1 model, hyperplastic islets in the LIRKO mice showed alterations in the adherens junction (FIG. 6C). Furthermore, the hyperplastic islets in LIRKOs also showed an absence of E-cadherin, pointing to a common pathway for β cell expansion that critically requires the presence of both copies of the PDX-1 gene. Consistent with earlier reports, no significant differences in non-β cells were observed among the groups (FIG. 6B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1) A method of increasing an initial population of mammalian β-cells that secrete insulin in response to glucose, the method comprising:
providing an initial population of fully-differentiated β-cells from a mammal;
contacting the cells with an exogenous modulator of E-cadherin signaling, in an amount and for a time sufficient to cause the cells to de-differentiate, wherein the modulator of E-cadherin signaling is selected from the group consisting of antibodies that bind selectively to E-cadherin and E-cadherin dominant negatives;
allowing the de-differentiated cells to proliferate; and
removing the modulator, to allow the de-differentiated cells to re-differentiate into β-cells that secrete insulin;
thereby increasing the initial population of mammalian-cells that secrete insulin in response to glucose.

2) The method of claim 1, wherein the mammal is a human.

3) The method of claim 1, wherein contacting the cells comprises administering or culturing the cells in the presence of the modulator of E-cadherin signaling.

4) The method of claim 1, wherein the modulator of E-cadherin signaling is a compound that inhibits E-cadherin signaling.

5) The method of claim 1, wherein the de-differentiated cells do not substantially secrete insulin in response to glucose.

6) The method of claim 1, wherein the de-differentiated cells are allowed to proliferate for a time sufficient to increase the population.

7) The method of claim 1, wherein removing the modulator comprises culturing/incubating the de-differentiated cells in the absence of the modulator, reducing the concentration or amount of the modulator, or ceasing administration of the modulator.

8) The method of claim 1, wherein the initial population of mammalian β-cells is in the pancreas of a living mammal, wherein contacting the cells with the modulator comprises administering a therapeutic composition comprising the modulator to the mammal.

9) The method of claim 8, wherein the therapeutic composition is administered locally into the pancreas of the mammal.

10) The method of claim 1, wherein the cells are derived from a human.

11) The method of claim 1, further comprising determining if the re-differentiated β-cells secrete insulin.

12) The method of claim 1, further comprising determining if the re-differentiated β-cells secrete insulin in a glucose-dependent manner.

13) The method of claim 1, further comprising placing the re-differentiated cells into a sterile preparation.

14) The method of claim 1, further comprising returning the re-differentiated cells to the mammal from which they came.

15) The method of claim 1, further comprising transplanting the re-differentiated cells to another mammal.

16) The method of claim 15, wherein the other mammal is of the same species.

17) A method of increasing a population of glucose-sensitive insulin secreting cells in a subject, the method comprising transplanting a population of redifferentiated cells produced by the method of claim 1 into the subject.

18) The method of claim 17, wherein the cells were originally derived from the subject.

19) A method of increasing a population of glucose-sensitive insulin secreting cells in a pancreas of a subject, the method comprising transiently administering to the subject an exogenous modulator of E-cadherin signaling, wherein the modulator is selected from the group consisting of antibodies that bind selectively to E-cadherin and E-cadherin dominant negatives.

20) The method of claim 19, wherein the modulator of E-cadherin signaling is a compound that inhibits E-cadherin signaling.

21) The method of claim 19, wherein the modulator is administered locally into the pancreas of the subject.
